SEQUENCE LISTING (16 sequences)

SEQ ID NO: 1 - DNA, 4942 bp, Unknown
  Nucleotide sequence of human corin
aaatcatccgtagtgcctccccgggggacacgtagaggagagaaaagcgaccaagataaaagtggacagaagaataagcgagactttttatccatgaaacagtctcctgccctcgctccggaagagcgctgccgcagagccgggtccccaaagccggtcttgagagctgatgacaataacatgggcaatggctgctctcagaagctggcgactgctaacctcctccggttcctattgctggtcctgattccatgtatctgtgctctcgttctcttgctggtgatcctgctttcctatgttggaacattacaaaaggtctattttaaatcaaatgggagtgaacctttggtcactgatggtgaaatccaagggtccgatgttattcttacaaatacaatttataaccagagcactgtggtgtctactgcacatcccgaccaacacgttccagcctggactacggatgcttctctcccaggggaccaaagtcacaggaatacaagtgcctgtatgaacatcacccacagccagtgtcagatgctgccctaccacgccacgctgacacctctcctctcagttgtcagaaacatggaaatggaaaagttcctcaagtttttcacatatctccatcgcctcagttgctatcaacatatcatgctgtttggctgtaccctcgccttccctgagtgcatcattgatggcgatgacagtcatggactcctgccctgtaggtccttctgtgaggctgcaaaagaaggctgtgaatcagtcctggggatggtgaattactcctggccggatttcctcagatgctcccagtttagaaaccaaactgaaagcagcaatgtcagcagaatttgcttctcacctcagcaggaaaacggaaagcaattgctctgtggaaggggtgagaactttctgtgtgccagtggaatctgcatccccgggaaactgcaatgtaatggctacaacgactgtgacgactggagtgacgaggctcattgcaactgcagcgagaatctgtttcactgtcacacaggcaagtgccttaattacagccttgtgtgtgatggatatgatgactgtggggatttgagtgatgagcaaaactgtgattgcaatcccacaacagagcatcgctgcggggacgggcgctgcatcgccatggagtgggtgtgtgatggtgaccacgactgtgtggataagtctgacgaggtcaactgctcctgtcacagccagggtctggtggaatgcagaaatggacaatgtatccccagcacgtttcaatgtgatggtgacgaggactgcaaggatgggagtgatgaggagaactgcagcgtcattcagacttcatgtcaagaaggagaccaaagatgcctctacaatccctgccttgattcatgtggtggtagctctctctgtgacccgaacaacagtctgaataactgtagtcaatgtgaaccaattacattggaactctgcatgaatttgccctacaacagtacaagttatccaaattattttggccacaggactcaaaaggaagcatccatcagctgggagtcttctcttttccctgcacttgttcaaaccaactgttataaatacctcatgttcttttcttgcaccattttggtaccaaaatgtgatgtgaatacaggcgagcatatccctccttgcagggcattgtgtgaacactctaaagaacgctgtgagtctgttcttgggattgtgggcctacagtggcctgaagacacagattgcagtcaatttccagaggaaaattcagacaatcaaacctgcctgatgcctgatgaatatgtggaagaatgctcacctagtcatttcaagtgccgctcaggacagtgtgttctggcttccagaagatgtgatggccaggccgactgtgacgatgacagtgatgaggaaaactgtggttgtaaagagagagatctttgggaatgtccatccaataaacaatgtttgaagcacacagtgatctgcgatgggttcccagactgccctgattacatggacgagaaaaactgctcattttgccaagatgatgagctggaatgtgcaaaccatgcgtgtgtgtcacgtgacctgtggtgtgatggtgaagccgactgctcagacagttcagatgaatgggactgtgtgaccctctctataaatgtgaactcctcttcctttctgatggttcacagagctgccacagaacaccatgtgtgtgcagatggctggcaggagatattgagtcagctggcctgcaagcagatgggtttaggagaaccatctgtgaccaaattgatacaggaacaggagaaagagccgcggtggctgacattacactccaactgggagagcctcaatgggaccactttacatgaacttctagtaaatgggcagtcttgtgagagcagaagtaaaatttctcttctgtgtactaaacaagactgtgggcgccgccctgctgcccgaatgaacaaaaggatccttggaggtcggacgagtcgccctggaaggtggccatggcagtgttctctgcagagtgaacccagtggacatatctgtggctgtgtcctcattgccaagaagtgggttctgacagttgcccactgcttcgaggggagagagaatgctgcagtttggaaagtggtgcttggcatcaacaatctagaccatccatcagtgttcatgcagacacgctttgtgaagaccatcatcctgcatccccgctacagtcgagcagtggtggactatgacatcagcatcgttgagctgagtgaagacatcagtgagactggctacgtccggcctgtctgcttgcccaacccggagcagtggctagagcctgacacgtactgctatatcacaggctggggccacatgggcaataaaatgccatttaagctgcaagagggagaggtccgcattatttctctggaacattgtcagtcctactttgacatgaagaccatcaccactcggatgatatgtgctggctatgagtctggcacagttgattcatgcatgggtgacagcggtgggcctcttgtttgtgagaagcctggaggacggtggacattatttggattaacttcatggggctccgtctgcttttccaaagtcctggggcctggcgtttatagtaatgtgtcatatttcgtcgaatggattaaaagacagatttacatccagacctttctcctaaactaattataaggatgatcagagacttttgccagctacactaaaagaaaatggccttcttgactgtgaagagctgcctgcagagagctgtacagaagcacttttcatggacagaaatgctcaatcgtgcactgcaaatttgcatgtttgttttggactaatttttttcaatttattttttcaccttcatttttctcttatttcaagttcaatgaaagactttacaaaagcaaacaaagcagactttgtccttttgccaggcctaaccatgactgcagcacaaaattatcgactctggcgagatttaaaatcaggtgctacagtaacaggttatggaatggtctcttttatcctatcacaaaaaaagacatagatatttaggctgattaattatctctaccagtttttgtttctcaagctcagtgcatagtggtaaatttcagtgttaacattggagacttgcttttctttttctttttttataccccacaattcttttttattacacttcgaattttagggtacacgagcacaacgtgcaggttagttacatatgtatacatgtgccatgttggtgtgctgaacccagtaactcgtcatttgatttattaaaagccaagataatttacatgtttaaagtatttactattacccccttctaatgtttgcataattctgagaactgataaaagacagcaataaaagaccagtgtcatccatttaggtagcaagacatattgaatgcaaagttctttagatatcaatattaacacttgacattattggaccccccattctggatgtatatcaagatcataattttatagaagagtctctatagaactgtcctcatagctgggtttgttcaggatatatgagttggctgattgagactgcaacaactacatctatatttatgggcaatattttgttttacttatgtggcaaagaactggatattaaactttgcaaaagagaatttagatgagagatgcaattttttaaaaagaaaattaatttgcatccctcgtttaattaaatttatttttcagttttcttgcgttcatccataccaacaaagtcataaagagcatattttagagcacagtaagactttgcatggagtaaaacattttgtaattttcctcaaaagatgtttaatatctggtttcttctcattggtaattaaaattttagaaatgatttttagctctaggccactttacgcaactcaatttctgaagcaattagtggtaaaaagtatttttccccactaaaaaactttaaaacacaaatcttcatatatacttaatttaattagtcaggcatccattttgccttttaaacaactaggattccctactaacctccaccagcaacctggactgcctcagcattccaaatagatactacctgcaattttatacatgtatttttgtatcttttctgtgtgtaaacatagttgaaattcaaaaagttgtagcaatttctatactattcatctcctgtccttcagtttgtataaacctaaggagagtgtgaaatccagcaactgaattgtggtcacgattgtatgaaagttcaagaacatatgtcagttttgttacagttgtagctacatactcaatgtatcaacttttagcctgctcaacttaggctcagtgaaatatatatattatacttattttaaataattcttaatacaaataaaatggtaatggtctaa

SEQ ID NO: 2 - protein, 1042 aa, Unknown
  Amino acid sequence of human corin
MKQSPALAPEERCRRAGSPKPVLRADDNNMGNGCSQKLATANLLRFLLLVLIPCICALVLLLVILLSYVGTLQKVYFKSNGSEPLVTDGEIQGSDVILTNTIYNQSTVVSTAHPDQHVPAWTTDASLPGDQSHRNTSACMNITHSQCQMLPYHATLTPLLSVVRNMEMEKFLKFFTYLHRLSCYQHIMLFGCTLAFPECIIDGDDSHGLLPCRSFCEAAKEGCESVLGMVNYSWPDFLRCSQFRNQTESSNVSRICFSPQQENGKQLLCGRGENFLCASGICIPGKLQCNGYNDCDDWSDEAHCNCSENLFHCHTGKCLNYSLVCDGYDDCGDLSDEQNCDCNPTTEHRCGDGRCIAMEWVCDGDHDCVDKSDEVNCSCHSQGLVECRNGQCIPSTFQCDGDEDCKDGSDEENCSVIQTSCQEGDQRCLYNPCLDSCGGSSLCDPNNSLNNCSQCEPITLELCMNLPYNSTSYPNYFGHRTQKEASISWESSLFPALVQTNCYKYLMFFSCTILVPKCDVNTGEHIPPCRALCEHSKERCESVLGIVGLQWPEDTDCSQFPEENSDNQTCLMPDEYVEECSPSHFKCRSGQCVLASRRCDGQADCDDDSDEENCGCKERDLWECPSNKQCLKHTVICDGFPDCPDYMDEKNCSFCQDDELECANHACVSRDLWCDGEADCSDSSDEWDCVTLSINVNSSSFLMVHRAATEHHVCADGWQEILSQLACKQMGLGEPSVTKLIQEQEKEPRWLTLHSNWESLNGTTLHELLVNGQSCESRSKISLLCTKQDCGRRPAARMNKRILGGRTSRPGRWPWQCSLQSEPSGHICGCVLIAKKWVLTVAHCFEGRENAAVWKVVLGINNLDHPSVFMQTRFVKTIILHPRYSRAVVDYDISIVELSEDISETGYVRPVCLPNPEQWLEPDTYCYITGWGHMGNKMPFKLQEGEVRIISLEHCQSYFDMKTITTRMICAGYESGTVDSCMGDSGGPLVCEKPGGRWTLFGLTSWGSVCFSKVLGPGVYSNVSYFVEWIKRQIYIQTFLLN

SEQ ID NO: 3 - DNA, 4890 bp, Unknown
  Nucleotide sequence of mouse corin
ctagtccccaggcagacggtccctcactcctgtggcttggcgtcggagacgctggcagtcatgggcagggtttccttcagcgttcgggtcagctccgtgcggagagcccgctgctcttgtcctgggcgatgctacctctcctgcagagtccctccaaccaccgccctccgtgcactgaacggtcttggctgcgcgggggttccgggggagactgcaggtggagccgtcggacccggccccttggggacccgtggcttcctctccgggtccaagttccaggctcccggcagctggaaggattgctttggagccccgcctgctccagacgtcttgagagcagacaggagcgtgggcgagggctgtcctcagaagctggtgactgctaacttgctgcgcttcctcctgctggtgctcatcccctgcatctgcgccctcatcgtgctgctggccatcctgctgtcctttgtgggaacattaaaaagggtttatttcaaatcaaatgacagtgaacctttggtcactgatggggaagctcgagtgcctggtgttattcctgtaaatacagtttattatgagaacacaggggcgccctctctgccccccagccagtccactccagcctggacaccgagagctccttctccagaggaccagagtcacaggaacacaagcacctgcatgaacatcactcacagccagtgtcaaattctgccctaccacagcacgttggcacctctcttgccaattgtcaaaaacatggacatggagaagttcctcaagttcttcacgtacctccatcgcctcagttgctatcaacatatcctgctcttcggctgtagcctcgccttccctgagtgcgttgttgatggcgatgacaggcatggtcttctaccctgtagatctttctgtgaggctgcaaaagaaggatgcgaatctgtcctgggaatggtgaactcctcctggccggattccctcagatgctctcagtttagggaccacactgagactaacagcagtgtcagaaagagctgcttctcactgcagcaggaacatggaaagcaatcactctgtggagggggcgagagcttcctgtgtaccagcgggctctgcgtccccaagaagctgcagtgtaacggctataatgactgtgatgactggagcgacgaggcgcattgcaactgcagcaaggatctgtttcactgtggcacaggcaagtgcctccattacagcctcttgtgtgatgggtacgatgactgtggggacctgagtgacgagcaaaactgtgattgtaatctcacaaaagagcatcgctgtggagatgggcgctgcattgcggctgagtgggtgtgcgatggggaccatgactgtgtggacaagtctgatgaggtcaactgctcttgtcacagccagggcctggtggaatgcagaagtggacagtgcatccctagcaccttccagtgtgatggggacgaagactgtaaggatgggagtgacgaggagaactgcagtgacagtcagacgccatgtccagaaggagaacagggatgccttggcagttcctgcgtcgaatcctgtgctggtagctctctgtgtgactcagacagcagcctgagtaactgcagtcaatgtgagcccatcactttggaactctgcatgaatttgccctacaaccatacacattatccaaattaccttggccacagaactcaaaaggaagcgtccatcagctgggagtcatcccttttccctgcccttgtacaaaccaactgttacaaatacctcatgtttttcgcttgcaccattttggttccaaagtgtgatgtgaatacaggacaacgcatcccgccttgcagactcctgtgtgagcactccaaagagcgctgtgagtctgttctgggaatcgttggcctgcagtggcctgaagacaccgactgcaatcaatttccagaggaaagttcagacaatcaaacttgcctcctgcccaatgaagatgtggaagaatgctctccgagtcacttcaaatgccgctcgggacgatgcgttctgggctccaggagatgtgacggccaggctgactgtgacgacgacagtgacgaggagaactgtggttgtaaagagagagctctttgggaatgtccatttaataagcaatgtctgaagcatacattaatctgcgatgggtttccagattgtccagacagtatggatgaaaaaaactgctcattttgccaagacaatgagctggaatgtgccaaccatgagtgtgtgccgcgtgacctttggtgcgacggatgggtcgactgctcagacagttctgatgaatggggctgtgtgaccctctctaaaaatgggaactcctcctcattgctgactgttcacaaatctgcaaaggaacaccacgtgtgtgctgacggctggcgggagacgttgagtcagctggcctgcaagcagatgggtttaggagaaccgtctgtgaccaagctgatcccaggacaggaaggccagcagtggctgaggttgtaccccaactgggagaatctcaatgggagcaccttgcaggagctgctggtatacaggcactcctgcccaagcagaagtgagatttcccttctgtgctccaagcaagactgtggccgccgccctgctgcccgaatgaacaagaggatccttgggggtcggactagtcgtcctgggaggtggccgtggcagtgctctctgcagagtgaacccagtggacatatctgtggctgtgtcctcattgccaagaagtgggtcctgacagttgcccattgctttgaagggagagaagacgctgatgtttggaaagtggtatttggcataaacaacctggaccatccatcaggcttcatgcagacccgctttgtgaagaccatcctgctacatccccgttacagtcgagcagtggtagactatgatatcagcgtggtggagctgagcgatgatatcaatgagacaagctacgtcagacctgtctgcctacccagtccggaggagtatctagaaccagatacgtactgctacatcacaggctggggccacatgggcaataaaatgccctttaagctgcaggagggagaggtccgcattatccctctggagcagtgccagtcctattttgacatgaagaccatcaccaatcggatgatctgtgctggctatgagtctggcaccgtggactcctgcatgggagacagcggtgggcctctggtttgtgaacgacccggaggacagtggacattatttggtttaacttcatggggctccgtctgcttttccaaagttctgggacctggagtgtacagcaatgtgtcttactttgtgggctggattgaaagacaaatatatatccagacctttctccaaaagaaatcccaaggataatcagagactttgtggggaaacctacatggagaatgaccctctgaaacagaagcttgtcctgccaagagctgtacgaacaggcgtttcacggacaggacgctcaacatgcaccgcaagatctctcctgtttgtgctagatgagttttactcaggctttaatctctttcaacattatcatttattaatttcatgaatccttttaaaagcacagagcaaagtaggttttgttattttgctaggctaaccttgaatgtagtgtgcaattatcaacccatagagacatttggagctctagggtaacaagttatagaaagctccttttattactactacaagacacacacggagatacacgctgactgatctccagtttctgcttaagcccagtggcttagggggcacatttcagaactgatcttggagactggcttttaatttgtagaaagccaagagaatatatatgcttttattatttactctactcttctaaataacttgaagaaatcatgaaagacagagaaaggacccacagtgttgatctagacagttgaagttgcaagaatgtaaaattctctagccaaccaaactaacactctgaagtaagtagaattctatcctttctgtattcaaattaagcttaaaatctccaccagatttgttcccgttactgggaattttcggagtatgtcacttagatgactgtgatgtcaaaagccaggtcaatccttgaggaaataatttgtttgcttatgtgggaatgaataagaatctttccattccgcaaaacacacaaattaaaaaggagaaaaaaattaaataacattccacacccaattaattctgaaaattagtctgcttgtattcacccaaaacagaaaagttacagaaatatatttcaaagtgcagcaaaatgttgcatggagtatataacattttgcaatttccccctcatgatgtctaacatccggtattgccatttgcctcattgataattaaaactaaattttaaggatgcttttaagcactgggccactttatgggaatcaatttccaaagcaattagtggttacaagtattttttcccactaaaaagtttcaaaacacaaaccttcatactaaattaattagccagacatgaactatgtaacatgcaaatgcctttttgaacaagtaggatgcactgctaaacttcaccagcaaccaaactgcctcagtattgcttacagggactacctgcaattttatatgtgtattttgtactctttttctagatagttcaaatgcaaaacattgtttcaacccctattctccatgttgttcacctcttgtcctggaatttgttacaaagtgtgtgtagcaaatgattgtactgcggtcaggactatatgaaggtttaggaccatcgggtcggttttgttataattgttggcacataattaataaaatatttttagcattggtaaaaaaaaaaaaaaaaaaaaaaaaaaa

SEQ ID NO: 4 - protein, 1113 aa, Unknown
  Amino acid sequence of mouse corin
MGRVSFSVRVSSVRRARCSCPGRCYLSCRVPPTTALRALNGLGCAGVPGETAGGAVGPGPLGTRGFLSGSKFQAPGSWKDCFGAPPAPDVLRADRSVGEGCPQKLVTANLLRFLLLVLIPCICALIVLLAILLSFVGTLKRVYFKSNDSEPLVTDGEARVPGVIPVNTVYYENTGAPSLPPSQSTPAWTPRAPSPEDQSHRNTSTCMNITHSQCQILPYHSTLAPLLPIVKNMDMEKFLKFFTYLHRLSCYQHILLFGCSLAFPECVVDGDDRHGLLPCRSFCEAAKEGCESVLGMVNSSWPDSLRCSQFRDHTETNSSVRKSCFSLQQEHGKQSLCGGGESFLCTSGLCVPKKLQCNGYNDCDDWSDEAHCNCSKDLFHCGTGKCLHYSLLCDGYDDCGDLSDEQNCDCNLTKEHRCGDGRCIAAEWVCDGDHDCVDKSDEVNCSCHSQGLVECRSGQCIPSTFQCDGDEDCKDGSDEENCSDSQTPCPEGEQGCLGSSCVESCAGSSLCDSDSSLSNCSQCEPITLELCMNLPYNHTHYPNYLGHRTQKEASISWESSLFPALVQTNCYKYLMFFACTILVPKCDVNTGQRIPPCRLLCEHSKERCESVLGIVGLQWPEDTDCNQFPEESSDNQTCLLPNEDVEECSPSHFKCRSGRCVLGSRRCDGQADCDDDSDEENCGCKERALWECPFNKQCLKHTLICDGFPDCPDSMDEKNCSFCQDNELECANHECVPRDLWCDGWVDCSDSSDEWGCVTLSKNGNSSSLLTVHKSAKEHHVCADGWRETLSQLACKQMGLGEPSVTKLIPGQEGQQWLRLYPNWENLNGSTLQELLVYRHSCPSRSEISLLCSKQDCGRRPAARMNKRILGGRTSRPGRWPWQCSLQSEPSGHICGCVLIAKKWVLTVAHCFEGREDADVWKVVFGINNLDHPSGFMQTRFVKTILLHPRYSRAVVDYDISVVELSDDINETSYVRPVCLPSPEEYLEPDTYCYITGWGHMGNKMPFKLQEGEVRIIPLEQCQSYFDMKTITNRMICAGYESGTVDSCMGDSGGPLVCERPGGQWTLFGLTSWGSVCFSKVLGPGVYSNVSYFVGWIERQIYIQTFLQKKSQG

SEQ ID NO: 5 - DNA, 22 bp, Unknown
  Sense primer used to amplify full-length human corin cDNA
agagaaaagcgaccaagataaa

SEQ ID NO: 6 - DNA, 22 bp, Unknown
  Antisense primer used to amplify full-length human corin cDNA
gtttaggagaaaggtctggatg

SEQ ID NO: 7 - DNA, 23 bp, Unknown
  Sense primer used to amplify human agouti-signaling protein
ggatggatgtcacccgcttactc

SEQ ID NO: 8 - DNA, 21 bp, Unknown
  Antisense primer used to amplify human agouti-signaling protein
gcagttgaggctgagcacgcg

SEQ ID NO: 9 - DNA, 23 bp, Unknown
  Sense primer used to amplify human agouti-related protein
atgctgaccgcagcggtgctgag

SEQ ID NO: 10 - DNA, 21 bp, Unknown
  Antisense primer used to amplify human agouti-related protein
ggtgcggctgcagggattcat

SEQ ID NO: 11 - DNA, 18 bp, Unknown
  Sense primer used to amply mouse agouti-related protein
caaaggccatgctgactg

SEQ ID NO: 12 - DNA, 23 bp, Unknown
  Antisense primer used to amplify mouse agouti-related protein
ggtgcgactacagaggttcgtgg

SEQ ID NO: 13 - DNA, 24 bp, Unknown
  Sense primer used to amplify human guanylin
tgccatgaatgccttcctgctctc

SEQ ID NO: 14 - DNA, 25 bp, Unknown
  Antisense primer used to amplify human guanylin
gcatccggtacaggcagcgtaggca

SEQ ID NO: 15 - DNA, 24 bp, Unknown
  Sense primer used to amplify human pro-atrial natriuretic peptide
agacagagcagcaagcagtggatt

SEQ ID NO: 16 - DNA, 25 bp, Unknown
  Antisense primer used to amplify human pro-atrial natriuretic peptide
gtaccggaagctgttacagcccagt

(19) United States
(12) Patent Application Publication
(10) Pub. No.: US 2011/0160128 A1
(43) Pub. Date: Jun. 30, 2011

(54) Corin for Treating Obesity and Diabetes

(76) Inventors: Qingyu Wu, Orange Village, OH (US); Jingjing Jiang, Cleveland, OH (US); Wei Wang, Richmond Heights, OH (US)

(21) Appl. No.: 12/935,228

(22) PCT Filed: Mar. 25, 2009
(86) PCT No.: PCT/US2009/038216

§ 371 Date: Jan. 11, 2011

Related U.S. Application Data
(60) Provisional application No. 61/072,222, filed on Mar. 28, 2008.

(51) Int. Cl. A61K 38/17; A61K 31/711; A61P 3/06

(52) U.S. Cl. 514/4.8; 514/44 R

(57) ABSTRACT

Provided herein are methods of inhibiting agouti or agouti-related protein (AGRP) in a cell or in an individual in need thereof, comprising administering to the cell or the individual an effective amount of an agent that induces corin expression, activity or a combination thereof in the cell or individual. The invention also provides a method of treating obesity in an individual in need thereof comprising administering to the individual an effective amount of an agent that enhances corin expression, activity or a combination thereof in the individual. Further provided is a method of treating diabetes type II in an individual in need thereof comprising administering to the individual an effective amount of an agent that enhances corin expression, activity or a combination thereof in the individual.

### RELATED APPLICATION(S)

This application claims the benefit of U.S. Provisional Application No. 61/072,222, filed on Mar. 28, 2008. The entire teachings of the above application(s) are incorporated herein by reference.

### BACKGROUND OF THE INVENTION

Presently, there are few effective strategies for treating obesity and conditions associated with obesity such as diabetes (e.g., diabetes type II). Thus, a need exists for improved methods of treating such conditions.

### SUMMARY OF THE INVENTION

It is known that agouti-related protein (AGRP) and agouti proteins are elevated in obese patients and that overexpression of agouti or AGRP cause obesity and diabetes. The results described herein show that corin, a type II transmembrane serine protease that is expressed primarily in the heart and is known to process the cardiac hormone pro-atrial natriuretic peptide (pro-ANP) to mature ANP, can be used as a strategy to degrade AGRP and agouti proteins. Thus, corin-based approaches, which use an endogenous mechanism that is designed to control food intake, can be used to treat obesity and diabetes.

Therefore, provided herein are methods of inhibiting agouti or agouti-related protein (AGRP) in a cell or in an individual in need thereof, comprising administering to the cell or the individual an effective amount of an agent that induces corin expression, activity or a combination thereof in the cell or individual.

The invention also provides a method of treating obesity in an individual in need thereof comprising administering to the individual an effective amount of an agent that enhances corin expression, activity or a combination thereof in the individual.

Further provided is a method of treating diabetes type II in an individual in need thereof comprising administering to the individual an effective amount of an agent that enhances corin expression, activity or a combination thereof in the individual.

In one embodiment, the agent is a polypeptide. In another embodiment, the polypeptide is all or a biologically active portion of a mammalian corin protein. In yet another embodiment, the polypeptide is all or a biologically active portion of a modified mammalian corin protein.

In other embodiments, the agent for use in the methods of the invention is a nucleic acid. In one embodiment, the nucleic acid encodes all or a biologically active portion of a mammalian corin protein. In a particular embodiment, the nucleic acid is operably linked to the corin promoter.

### BRIEF DESCRIPTION OF THE DRAWINGS

FIG. 1 shows the melanocortin-mediated pathway. Alpha-melanocyte stimulating hormone (α-MSH) regulates pigmentation and energy homeostasis. In skin melanocyte, α-MSH binds to the melanocorin 4 receptor (Mc1r) and promotes eumelanin production. The binding of α-MSH to Mc1r is blocked by agouti or agouti-signaling protein (ASIP) peptides, which leads to pheomelanin production. In the central nervous system, α-MSH binds to the melanocorin 4 receptor (Mc4r) on neurons in hypothalamus, which inhibits appetite and food intake. Agouti and agouti-related protein (AGRP) block α-MSH binding to Mc4r, which promotes food intake and increases body weight. In agouti yellow mice (A^{y}), overexpression of agouti protein causes obesity, diabetes, and hypertension.

FIG. 2 shows that corin promotes mouse AGRP and human ASIP protein degradation. Top panel. HEK 293 cells were transfected with plasmids expressing mouse AGRP (mAGRP) and human ASIP (hASIP) together with a corin expressing plasmid or a control vector. As a positive control, the cells were transfected with a plasmid expressing human pro-ANP together with a corin expressing plasmid or a control vector. Pro-ANP, mAGRP, hASIP, and their derivatives in the conditioned media were analyzed by immunoprecipitation and Western analysis. Lower panel. The transfected cells were lysed. The expression of recombinant corin, pro-ANP, mASRP, and hASIP proteins in cell lysate were analyzed by Western blotting using an anti-V5 tag antibody. Bands representing corin, pro-ANP, hASIP, and mAGRP are indicted by arrows.

FIG. 3 shows that corin promotes mouse AGRP and human ASIP protein degradation. The experiments described in FIG. 2 were repeated and similar results were obtained. Top panel. HEK 293 cells were transfected with plasmids expressing mouse AGRP (mAGRP) and human ASIP (hASIP) together with a corin expressing plasmid or a control vector. As a positive control, the cells were transfected with a plasmid expressing human pro-ANP together with a corin expressing plasmid or a control vector. Pro-ANP, mAGRP, hASIP, and their derivatives in the conditioned media were analyzed by immunoprecipitation and Western analysis. Lower panel. The transfected cells were lysed. The expression of recombinant corin, pro-ANP, mASRP, and hASIP proteins in cell lysate were analyzed by Western blotting using an anti-V5 tag antibody. Bands representing corin, pro-ANP, hASIP, and mAGRP are indicted by arrows.

FIG. 4 shows that corin promotes human AGRP protein degradation. Top panel. HEK 293 cells were transfected with plasmids expressing human guanylin (negative control), pro-ANP (positive control), and AGRP together with a corin expressing plasmid or a control vector. Guanylin, pro-ANP, hAGRP, and their derivatives in the conditioned media were analyzed by immunoprecipitation and Western analysis using an anti-V5 tag antibody. Lower panel. The transfected cells were lysed. The expression of recombinant corin, guanylin, pro-ANP, and hAGRP proteins in cell lysate were analyzed by Western blotting using an anti-V5 tag antibody. Bands representing corin, guanylin, pro-ANP, and hAGRP are indicted by arrows.

FIG. 5 shows that corin degrades agouti and AGRP proteins and prevents overeating and obesity. The α-MSH-mediated pathway is described in the FIG. 1 legend. The data provided herein indicate that corin promotes the degradation of agouti/ASIP and AGRP proteins, thereby preventing overeating and obesity.

FIG. 6 shows how corin can be used as a therapy for obesity and diabetes. In mice, overexpression of agouti or AGRP proteins causes obesity, diabetes, and hypertension. In mice and humans, mutations in Mc4r cause obesity and diabetes. The data described herein show that corin promotes the degradation of agouti/ASIP and AGRP proteins, indicating that corin can be used as therapeutic agent to treat obesity and diabetes.

FIGS. 7A-7B is the nucleotide sequence of human corin (SEQ ID NO: 1).

FIG. 8 is the amino acid sequence of human corin (SEQ ID NO: 2).

FIGS. 9A-9B is the nucleotide sequence of mouse corin (SEQ ID NO: 3).

FIG. 10 is the amino acid sequence of mouse corin (SEQ ID NO: 4).

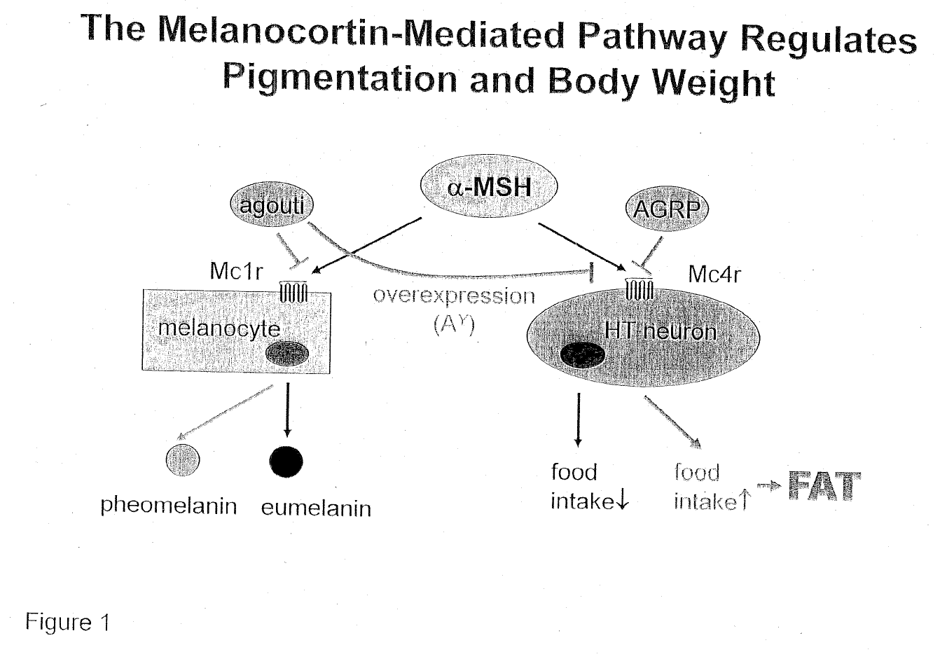

### DETAILED DESCRIPTION OF THE INVENTION

The melanocorin-mediate pathway plays an important role in energy homeostasis. In the central nervous system, alpha-melanocyte stimulating hormone (a-MSH) binds to the melanocorin 4 receptor (MC4r) and inhibits appetite, thereby reducing food intake. Naturally occurring MC4r mutations cause severe childhood-onset obesity.

Agouti and agouti-related protein (AGRP) are inhibitors of the melanocorin pathway, blocking α-MSH binding to MC4r. In mice, overexpression of agouti or AGRP cause obesity, diabetes and hypertension. In mice, agouti protein also regulates coat color formation.

A recent study suggested that corin may be involved in coat color determination but the mechanism is not known (Enshell-Seijffers, D., et al., Development, 135:217-225 (2008)). Described herein are experiments investigating whether corin, as a protease, is involved in the melanocorin pathway by degrading agouti and AGRP. Experiments performed show that corin indeed degraded recombinant mouse and human AGRP and human agouti proteins. Consistently, corin null mice are fatter than wild type controls (Chan, J., et al., PNAS, 102:785-790 (2005)). The increase of body weight in corin null mice is agouti gene-dependent. Thus, described herein are corin substrates, i.e., agouti and AGRP.

It is known that AGRP and agouti proteins are elevated in obese patients and that overexpression of agouti or AGRP cause obesity and diabetes. The results described herein show that corin can be used as a strategy to degrade AGRP and agouti proteins. Thus, corin-based approaches, which use an endogenous mechanism that is designed to control food intake, can be used to treat obesity and diabetes.

Accordingly, the invention provides for methods of inhibiting agouti or agouti-related protein (AGRP) in a cell or in an individual in need thereof, comprising administering to the cell or the individual an effective amount of an agent that induces corin expression, activity or a combination thereof in the cell or individual.

The invention also provides for methods of treating obesity in an individual in need thereof comprising administering to the individual an effective amount of an agent that induces corin expression, activity or a combination thereof in the individual, wherein the corin degrades agouti and/or AGRP in the individual thereby treating the obesity.

In addition, the invention provides for methods of treating diabetes (e.g., diabetes type II) in an individual in need thereof comprising administering to the individual an effective amount of an agent that induces corin expression, activity or a combination thereof in the individual, wherein the corin degrades agouti and/or AGRP in the individual thereby treating the diabetes.

Corin is a type II transmembrane serine protease of the trypsin superfamily having the following structurally distinct domains: a transmembrane/signal peptide, frizzled domains, low density lipoprotein receptor repeats (LDLR), scavenger receptor cysteine-rich repeats (SRCR) and a serine protease catalytic domain. Human corin is comprised of 1042 amino acids (SEQ ID NO: 2) which include a cytoplasmic tail at its N-terminus (amino acids 1 to 45 of SEQ ID NO: 2) followed by a transmembrane domain (amino acids 46 to 66 of SEQ ID NO: 2), a stem region composed of two frizzled-like cysteine-rich domains (CRD, amino acids 134 to 259 and 450 to 573 of SEQ ID NO: 2), eight low density lipoprotein receptor repeats (LDLR, amino acids 268 to 415 and 579 to 690 of SEQ ID NO: 2), a macrophage receptor-like domain (SRCR, amino acids 713 to 800 of SEQ ID NO: 2) and a serine protease catalytic domain at its C-terminus (CAT, amino acids 802 to 1042 of SEQ ID NO: 2). Amino acids 801 through 805 of SEQ ID NO: 2 (i.e., ArgIleLeuGlyGly or RILGG) is a conserved activation cleavage site, in which proteolytic cleavage of the peptide bind between Arg801 and Ile802 generates a catalytically active corin. See U.S. Pat. No. 6,806,075; U.S. Pat. No. 7,176,013; PCT Published Application No. WO 03/102135; and Wu, Q, Frontiers in Bioscience, 12:4179-4190 (2007) all of which are incorporated herein by reference.

As used herein, “induces” and “inducement” refers to enhancement of the expression and/or activity of all or a biologically active portion of a corin polypeptide which is being expressed either at normal or below normal levels in an individual or cell. Inducement of a corin polypeptide also includes the turning on of all or a biologically active portion of a corin polypeptide that is not being expressed in the cell or individual.

In the methods of the invention, corin polypeptide expression, activity or a combination thereof is increased in the individual or cell after administration of the agent compared to corin protein expression, activity of a combination thereof in the individual or cell prior to administration of the agent.

As used herein, “treating” or “treatment” refers to prevention of the condition (e.g., obesity, diabetes type II) or alleviation of the condition or some or all of the symptoms of the condition. Any agent or physiological stimulus that, when administered, causes corin to be active in the cell or individual, and thereby prevents or alleviates the dysfunction (e.g., obesity; diabetes) by inhibiting or degrading agouti or agouti-related protein (AGRP) in the cell or individual in need thereof, can be used in the methods of the invention. For example, any agent that induces corin expression, activity or a combination thereof in a cell or individual can be used in the methods provided herein. Example of such agents include peptides, nucleic acids (e.g., DNA, RNA), peptidomimetics, small molecules such as small organic molecules or other drugs which induce (partially, completely) corin expression, activity or a combination thereof.

In one embodiment, the agent is a polypeptide (also referred to herein as a protein). For example, the polypeptide can be a corin polypeptide or a biologically active portion thereof. In a particular embodiment, the corin polypeptide or biologically active portion thereof is a mammalian corin polypeptide such as a primate (e.g., human) corin, a murine (e.g., mouse, rat) corin, a feline corin, a canine corin, a bovine corin and the like. In one embodiment, the polypeptide is all or a biologically active portion of SEQ ID NO:2. In another embodiment, the polypeptide is all or a biologically active portion of SEQ ID NO: 4.

As used herein, a “biologically active portion of a corin polypeptide” is a portion of a corin polypeptide that retains the ability to degrade agouti or AGRP in a cell or individual. Examples of a biologically active portion of a corin polypeptide comprises at least one frizzled domain of the corin protein, at least one low density lipoprotein receptor (LDLR) repeat of the corin protein, a serine protease catalytic domain of the corin protein or a combination thereof. In one embodiment, the biologically active portion is a soluble corin polypeptide that lacks all, or substantially all, of the transmembrane domain. For example, a soluble corin polypeptide can include all or a portion of the extracellular domain. In one embodiment, the biologically active portion of the mammalian corin protein is a soluble corin polypeptide comprising all or a portion of the corin extracellular domain (e.g., from about amino acid 67 to about amino acid 1042 of SEQ ID NO: 2) (e.g., see U.S. Pat. No. 6,806,075 which is incorporated herein by reference). In another embodiment, the biologically active portion comprises a serine protease catalytic domain of a corin polypeptide. In yet another embodiment, the biologically active portion of the mammalian corin protein is a soluble corin polypeptide comprising the serine protease catalytic domain (e.g., from about amino acid 802 to about amino acid 1042 of SEQ ID NO: 2 (see U.S. Pat. No. 6,806,075 which is incorporated herein by reference).

The polypeptide for use in the methods of the invention can also be a modified corin polypeptide or a biologically active portion thereof that retains the ability to degrade agouti or AGRP in a cell or individual in need thereof. Such modifications include the deletions and/or substitutions (e.g., conservative, non-conservative) of amino acids in a wild type corin polypeptide sequence. Thus, as used herein, a “modified corin polypeptide” is a corin polypeptide that has been modified, and thus differs from the wild type corin but retains the wild type corin function of degrading agouti or AGRP in a cell or individual. Modified corin polypeptides encompasses corin sequence variants (e.g., allelelic variants) and polypeptides derived from other organisms, and which have substantial homology to a polypeptide encoded by a nucleic acid molecule comprising the nucleotide sequence of SEQ ID NOs: 1 or 3, and complements and portions thereof, or having substantial homology to a polypeptide encoded by a nucleic acid molecule comprising the nucleotide sequences of SEQ ID NO: 1 or 3. Modified corin polypeptides also include polypeptides substantially homologous or identical to corin polypeptides but derived from another organism, i.e., an ortholog; polypeptides that are substantially homologous or identical to these polypeptides that are produced by chemical synthesis; and polypeptides that are substantially homologous or identical to corin polypeptides that are produced by recombinant methods.

As used herein, two polypeptides (or a region of the polypeptides) are substantially homologous or identical when the amino acid sequences are at least about 82%, 85%, 90%, 92%, 94%, 95%, 96%, 97%, 98% or 99% homologous or identical. A substantially identical or homologous amino acid sequence, according to the present invention, will be encoded by a nucleic acid molecule hybridizing to SEQ ID NOs: 1 or 3, or portions thereof, under stringent conditions as more particularly described herein.

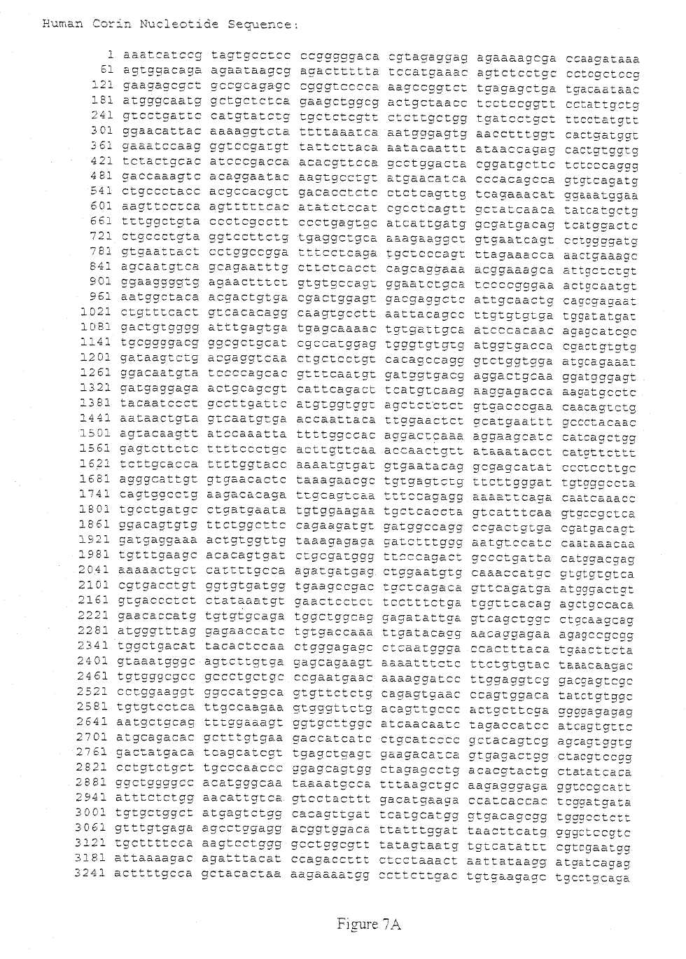

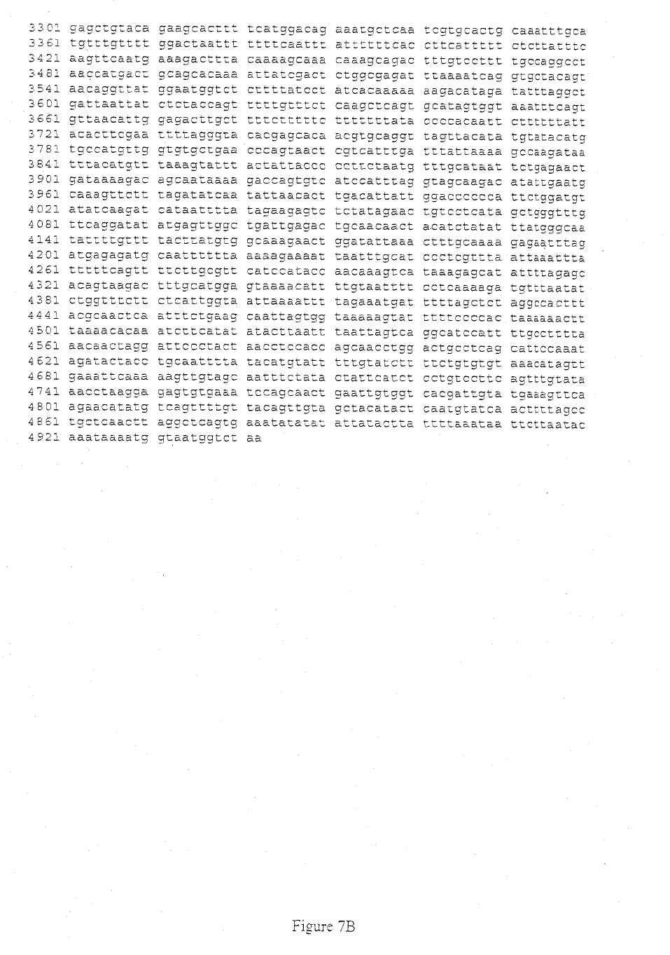

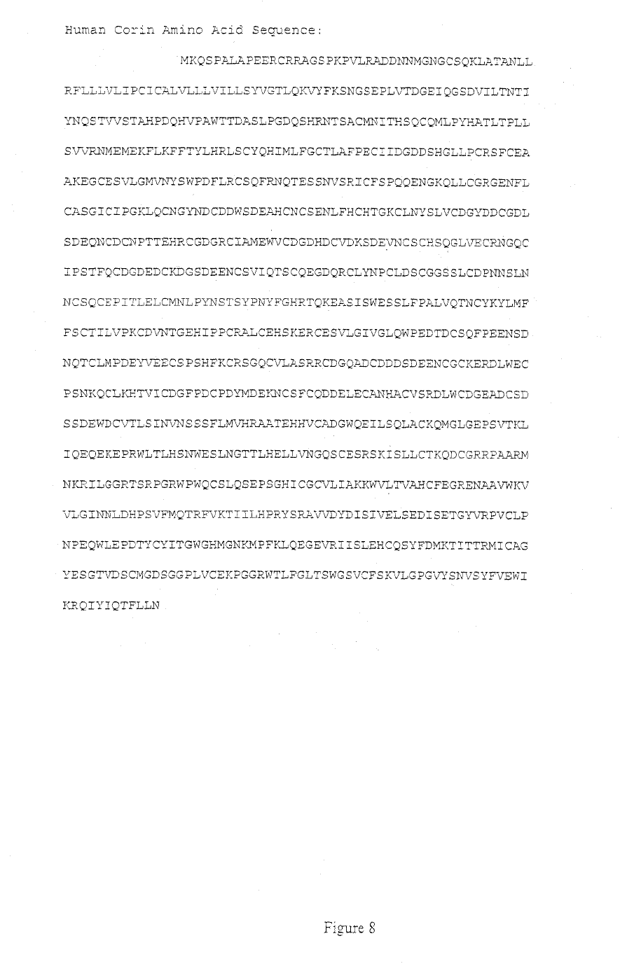

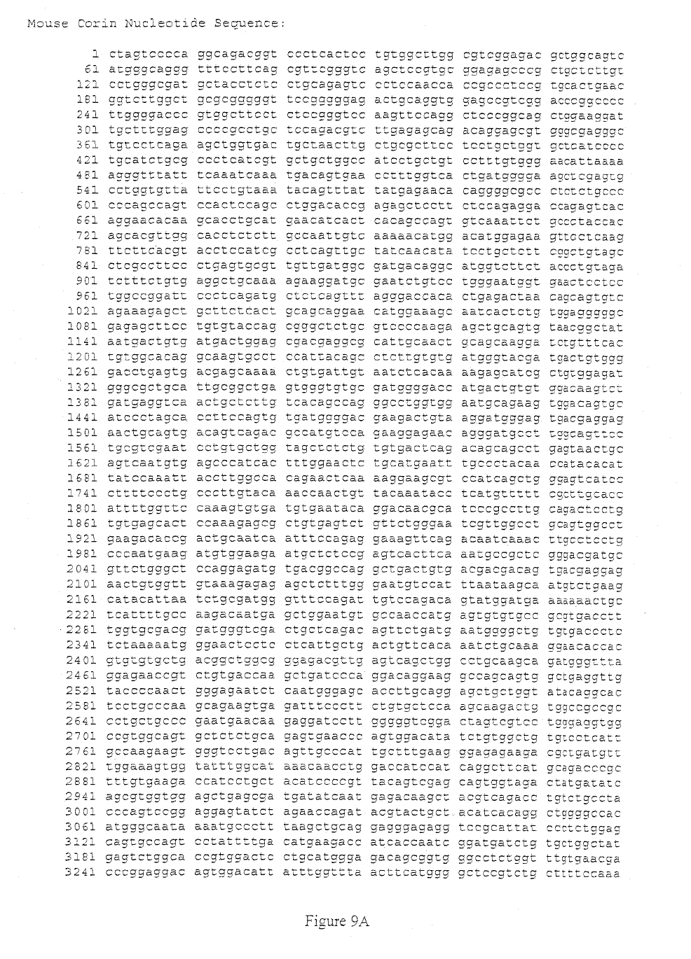

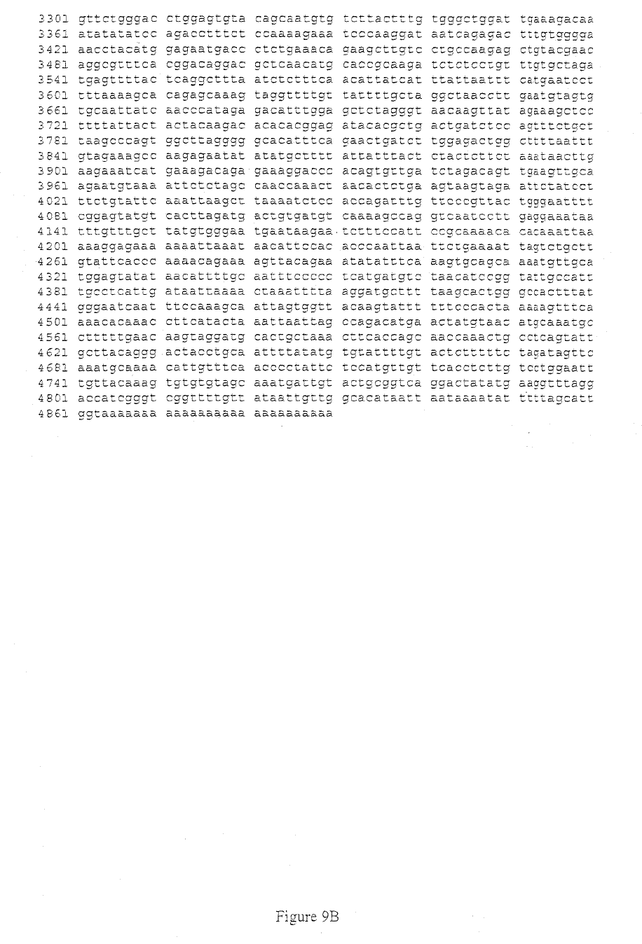

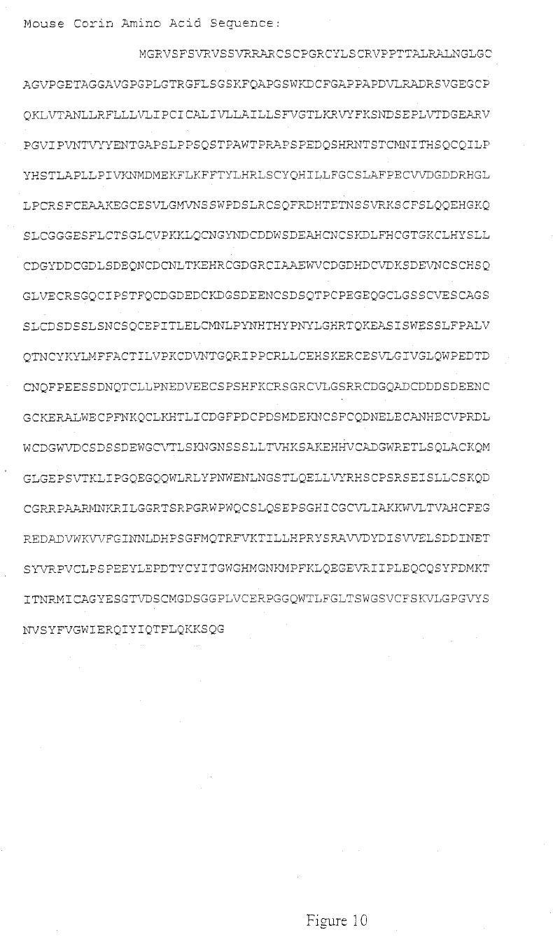

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of the amino acid or nucleotide sequence aligned for comparison purposes is at least 30%, 40%, 50%, 60%, 70%, 80%, 85%, 90%, 92%, 94%, 95%, 96%, 97%, 98%, 99% or 100% of the length of the reference sequence, for example, those sequences provided in FIGS. 7 and 9. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al., Nucleic Acids Res., 29:2994-3005 (2001), When utilizing BLAST and Gapped BLAST programs, the default parameters of the respective programs (e.g., BLASTN) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1. In another embodiment, the percent identity between two polypeptides or two polynucleotides is determined over the full-length of the polypeptide or polynucleotide of interest.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package (Accelrys, San Diego, Calif.). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci., 10: 3-5 (1994); and FASTA described in Pearson and Lipman, Proc. Natl. Acad. Sci USA, 85: 2444-8 (1988).

In another embodiment, the percent identity between two amino acid sequences can be accomplished using the GAP program in the GCG software package using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

Modified mammalian corin polypeptides include, for example, a corin polypeptide or biologically active portion thereof having an activation sequence that differs from the activation sequence of a wild type corin polypeptide (i.e., RILGG) and biologically active fragments thereof. A variety of such modified corin polypeptides which can be used in the methods provided herein are described in U.S. Pat. No. 7,176,013 which is incorporated herein by reference (e.g., SolCorin-EK and SolCorinPD-EK of U.S. Pat. No. 7,176,013). In a particular embodiment, the modified corin protein or biologically active portion thereof comprises a serine protease recognition sequence other than the corin serine protease recognition sequence. In another embodiment, the modified corin polypeptide or biologically active portion thereof comprises the corin extracellular domain and a protease recognition sequence other than the corin serine protease recognition sequence. Examples of protease recognition that can be used to replace the corin serine protease recognition sequence include a protease recognition sequence cleaved by a proteolytic enzyme selected from the group consisting of: enterokinase, thrombin, factor Xa, furin, PC1, PC2, PC5, PACE4 and a combination thereof. In a particular embodiment, the wild type corin activation sequence is replaced with the activation sequence DDDDK. In another embodiment, the wild type corin activation sequence is replaced with the activation sequence DDDDKILGG. The modified corin polypeptide or biologically active portion thereof can also comprise the corin serine protease catalytic domain and a protease recognition sequence other than the corin serine protease recognition sequence. Use of such modified corin polypeptides in the methods described herein can further comprise administering to the cell or individual the proteolytic enzyme which cleaves the modified corin polypeptide, thereby rendering the corin active.

A variety of methods for preparing a corin polypeptide, a modified corin polypeptide or a biologically active portion thereof are provided herein and known in the art. See, for example, U.S. Pat. No. 6,806,075 and U.S. Pat. No. 7,176,013).

Likewise, a variety of methods for determining the biological activity (determining whether such polypeptides retain the ability to degrade agouti or AGRP in a cell or individual in need thereof) of a corin polypeptide, a modified corin polypeptide or a biologically active portion thereof are known in art and provided herein. Examples of suitable assays which can be used to determine whether a portion of a corin polypeptide degrades agouti and/or AGRP include Western blotting assay enzyme-linked immunosorbent assay (ELISA), high performance liquid chromatography (HPLC), liquid chromatography mass spectrometry (LC-MS) and radioimmunoassays (RIA).

The agent for use in the methods of the invention can also be a nucleic acid. In one embodiment, the nucleic acid encodes all or a biologically active portion of a corin polypeptide (e.g., a mammalian corin polypeptide) and/or a modified corin polypeptide as described herein. In a particular embodiment, the nucleic acid is operably linked to a corin promoter.

In embodiments in which nucleic acid is used in the methods of the invention, the nucleic acid can be administered, for example, as naked DNA or in an expression vector. Examples of suitable expression vectors include plasmids and viral vectors (e.g., replication competent viral vectors; replication impaired viral vectors). Examples of suitable vectors that can be used in the methods of the invention include adenoviral vectors, lentiviral vectors, and poxviral vectors.

Alternatively, transcription of an endogenous corin gene (e.g., a silent endogenous corin gene) can be modulated. Any small molecule or physiological stimulus that affects the amounts or activity of the different transcription factors that modulate corin gene expression will affect levels of corin mRNA and protein. Alternatively, DNA based manipulations can be performed to change the regulation of the endogenous corin gene. Exogenous regulatory sequences can be added to the endogenous corin gene, putting the gene under the control of different DNA sequences, proteins that bind those sequences, and effectors that affect those proteins. In these situations, modulation of expression of corin occurs when the effector is administered from an external source or withheld, depending on the action that occurs at the regulation site.

Another manner of changing or modulating the amount of corin polypeptide in the individual is using transgenic technology. A transgene that encodes a desired corin polypeptide, modified corin polypeptide or biologically active portion thereof is inserted into the genome of the individual. The transgene can be inserted using recombinant techniques recognized and known to skilled persons such as molecular biologists. The transgenic individual can contain one or more copies of the transgene that encodes the corin polypeptide, the modified corin polypeptide or biologically active portion thereof. This transgene may contain the endogenous corin gene, a corin gene of another animal species, a modified corin gene or a biologically active portion thereof. In either instance, the transgene can be under the control of either endogenous regulation sites or regulation sites obtained from exogenous sources. Endogenous regulation sites can be employed when the transgene is inserted at an appropriate locus in the genome where gene expression is controlled by the endogenous regulation site. The regulation sites are often easier to include with the transgenes when the genome insertions are performed. In either situation, the inserted transgene encoding the desired corin polypeptide, modified corin polypeptide or biologically active portion thereof provides more control of the induction of the expression of the corin polypeptide. This increased control provides for the ability to alleviate obesity and diabetes.

In another embodiment, the nucleic acid can be a regulatory region that controls expression of a corin polypeptide, a modified corin polypeptide or a biologically active portion thereof. In a particular embodiment, the nucleic acid for use in the methods of the invention is an isolated polynucleotide comprising a corin expression control region as described in PCT Published Application No. WO 03/102135 which is incorporated herein by reference.

The corin polypeptide or nucleic acid for use in the methods of the invention can be isolated from natural sources (e.g., from a biological sample such as cells, tissues, fluids, organisms) or from cell lines (e.g., transformed cells) using known techniques such as detergent extraction, ammonium sulfate or ethanol precipitation, and/or chromatography. Alternatively, the corin polypeptide can be chemically synthesized or recombinantly produced using known techniques.

As used herein, a polypeptide or nucleic acid is said to be “isolated,” “substantially pure,” or “substantially pure and isolated” when it is substantially free of cellular material, when it is isolated from recombinant or non-recombinant cells, or free of chemical precursors or other chemicals when it is chemically synthesized. In addition, a polypeptide can be joined to another polypeptide with which it is not normally associated in a cell (e.g., in a “fusion protein”) and still be “isolated,” “substantially pure,” or “substantially pure and isolated.” An isolated, substantially pure, or substantially pure and isolated polypeptide or nucleic acid may be obtained, for example, using affinity purification techniques, hybridization techniques as well as other techniques described herein and known to those skilled in the art.

The (one or more) agent used to induce (e.g., enhance) corin expression and/or activity can be administered in a therapeutically effective amount (i.e., an amount that is sufficient to treat the condition or disease, such as by ameliorating symptoms associated with the condition or disease, preventing or delaying the onset of the condition or disease, and/or also lessening the severity or frequency of symptoms of the condition or disease). The amount that will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The one or more agents can be delivered in a composition, as described above, or by themselves. They can be administered systemically, or can be targeted to a particular tissue. The agent can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. A combination of any of the above methods of treatment can also be used.

In a particular embodiment, the agent is a pharmaceutical agent. The agent for use in the methods described herein can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In another embodiment, the invention is directed to agents which induce corin expression and/or activity for use as a medicament in therapy. For example, the agents identified herein can be used in the treatment of obesity and/or diabetes (e.g., diabetes type II). In addition, the agents identified herein can be used in the manufacture of a medicament for the treatment of obesity and/or diabetes (e.g., diabetes type II).

In the methods of the invention, the individual is typically a mammal. For example, the individual can be a a primate (e.g., human) corin, a murine (e.g., mouse, rat) corin, a feline corin, a canine corin, a bovine corin and the like. In a particular embodiment, the mammal is a human.

### EXEMPLIFICATION

### Methods and Materials

### Expression Plasmid Vectors

The full-length human corin cDNA was amplified from a heart cDNA library (BioChain, catalog# C1244122-10) in a PCR using Pfu polymerase (Stratagene, La Jolla, Calif.) and the following oligonucleotide primers: sense primer, 5′-AGA GAA AAG CGA CCA AGA TAA A-3′ (SEQ ID NO: 5) and antisense primer, 5′-GTT TAG GAG AAA GGT CTG GAT G-3′ (SEQ ID NO: 6). The cDNA fragment was cloned into pcDNA3.1-V5-6×His TOPO vector (Invitrogen). The resultant plasmid, pcDNAhCorin, encoded recombinant human corin protein containing a viral V5 tag at its C-terminus, which allowed the detection by an anti-V5 antibody (Invitrogen) in immunoprecipitation and Western analyses.

Full-length cDNAs for human agouti-signaling protein (ASIP), human agouti-related protein (AGRP), and mouse AGRP were purchased from Open Biosystems (catalog #MHS4426-98361263, MHS4426-98360814, and MMM1013-9497952, respectively). The cDNAs were amplified by PCR using Pfu polymerase and the following oligonucleotide primers: human ASIP sense primer, 5′-GGA TGG ATG TCA CCC GCT TAC TC-3′ (SEQ ID NO: 7) and antisense primer, 5′-GCA GTT GAG GCT GAG CAC GCG-3′ (SEQ ID NO: 8); human AGRP sense primer, 5′-ATG CTG ACC GCA GCG GTG CTG AG-3′ (SEQ ID NO: 9) and antisense primer, 5′-GGT GCG GCT GCA GGG ATT CAT-3′ (SEQ ID NO: 10); and mouse AGRP sense primer 5′-CAA AGG CCA TGC TGA CTG-3′ (SEQ ID NO: 11) and antisense primer 5′-GGT GCG ACT ACA GAG GTT CGT GG-3′ (SEQ ID NO: 12). The cDNA fragment was cloned into pcDNA3.1-V5-6×His TOPO vector (Invitrogen). The resultant plasmid, pcDNAhASIP, pcDNAhAGRP, and pcDNAmAGRP encode recombinant proteins containing a viral V5 tag at its C-terminus, which allowed the detection by an anti-V5 antibody in immunoprecipitation and Western analyses.

Full-length cDNAs for human guanylin and pro-atrial natriuretic peptide (pro-ANP) were amplified from human small intestine (BioChain, catalog #C1234226) and heart cDNA (BioChain, catalog #C 1244122) libraries, respectively. Oligonucleotide primers used were: human guanylin sense primer, 5′-TGC CAT GAA TGC CTT CCT GCT CTC-3′(SEQ ID NO: 13) and antisense primer, 5′-GCA TCC GGT ACA GGC AGC GTA GGC A-3′ (SEQ ID NO: 14) and human pro-ANP sense primer, 5′-AGA CAG AGC AGC AAG CAG TGG ATT-3′ (SEQ ID NO: 15) and antisense primer, 5′-GTA CCG GAA GCT GTT ACA GCC CAG T-3′ (SEQ ID NO: 16). The cDNA fragments were cloned into pcDNA3.1/V5 vector. These plasmids were used as negative and positive controls, respectively, in this study.

### Transfection and Western Analysis of AGRP and ASIP Degradation

HEK 293 cells were transfected with expression plasmids using LipofectAMINE 2000 (Invitrogen) according to manufacturer's instruction. Conditioned medium was collected after 16 to 24 h, and subjected to centrifugation at 13,000 rpm to remove cell debris. AGRP, ASIP, and their derivatives in the conditioned medium were analyzed by immunoprecipitation and Western blotting using an anti-V5 antibody that recognized the V5 tag attached to the C-termini of the recombinant peptides. The methods were used previously for analyzing pro-ANP processing, as described previously (Wu F, et al. JBC 2002; 277:16900-16905; Wu C, et al JBC 2003; 278:25847-25852; Liao X, et al. JBC 2007; 282:27728-27735).

To analyze recombinant proteins in cell lysate, transfected cells were lysed in a buffer containing 50 mM Tris-HCl, pH 8.0, 150 mM NaCl, 1% Triton X-100 (v/v), 10% glycerol (v/v), 1 mM phenylmethylsulfonyl fluoride, and a protease inhibitor cocktail (1:100 dilution, Sigma). Protein samples were mixed with a loading buffer with 2% β-mercaptoethanol and boiled at 100° C. for 5 min before being loaded onto an SDS-PAGE gel. Western analysis of recombinant proteins was done using an anti-V5 antibody, as described previously (Wu F, et al. JBC 2002; 277:16900-16905; Wu C, et al JBC 2003; 278:25847-25852; Liao X, et al. JBC 2007; 282:27728-27735).

### Results

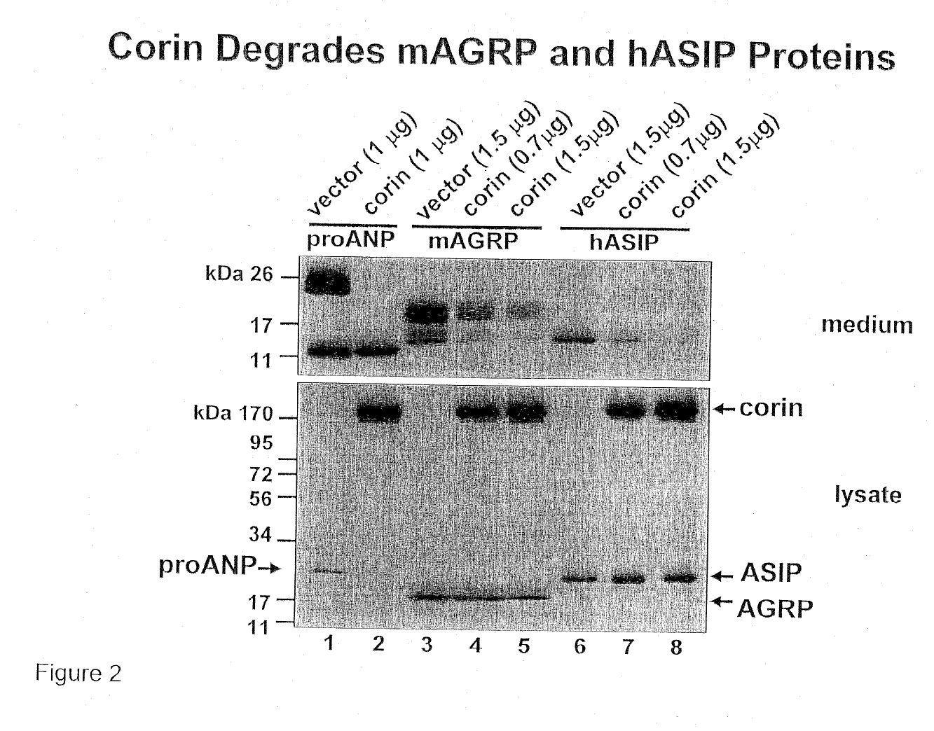

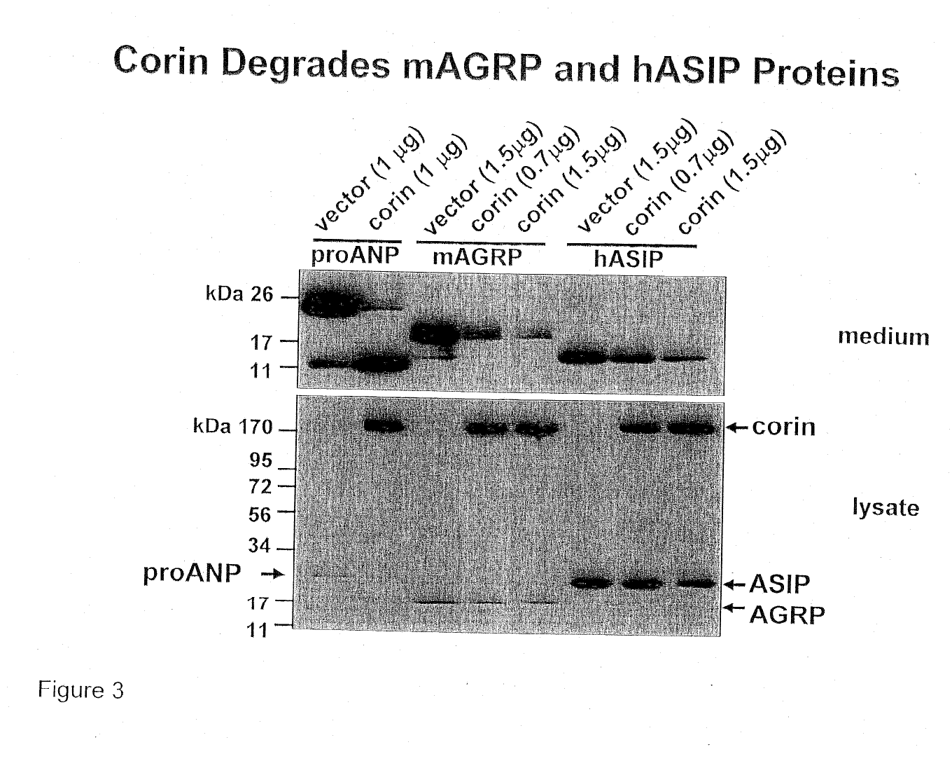

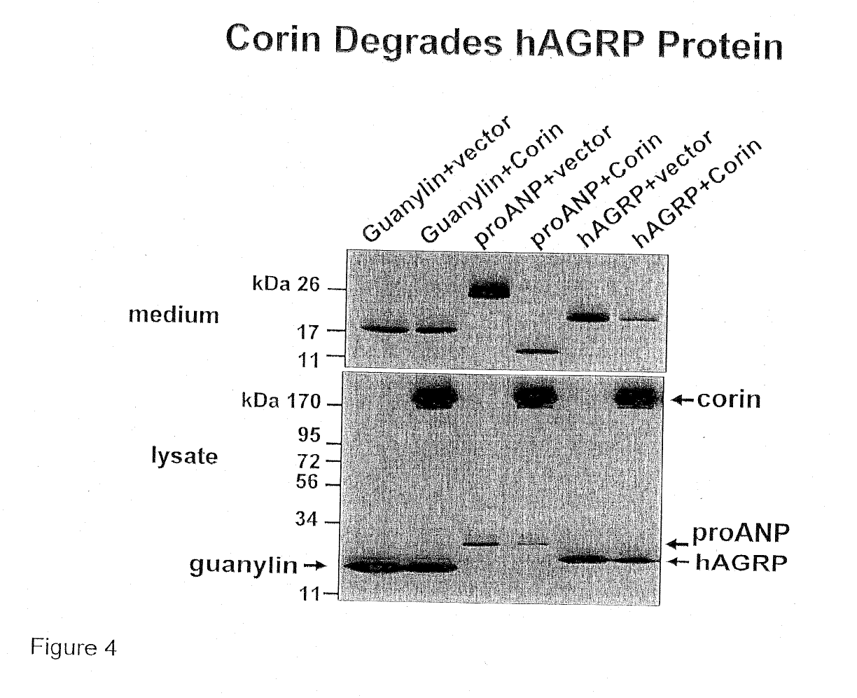

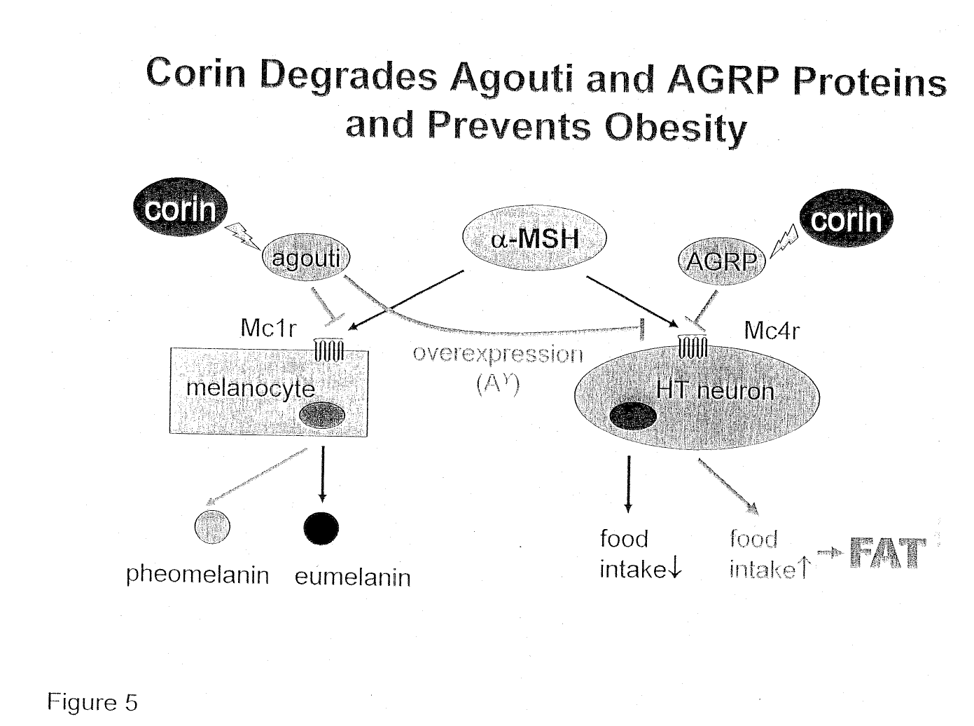

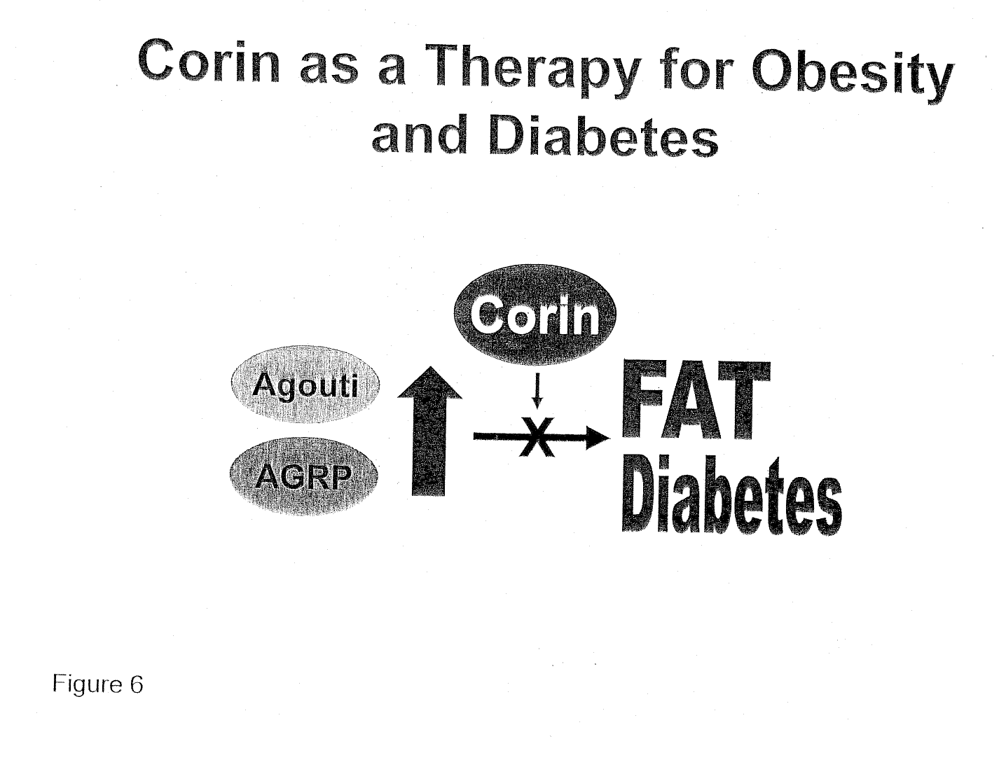

Recombinant mouse (FIGS. 2 and 3) and human (FIG. 4) AGPR proteins were expressed HEK 293 cells. On Western blots, the proteins appeared as bands of ˜19 kDa in samples from cell lysate (FIGS. 2-4, lower panels). In the conditioned medium, there were several bands for mouse AGRP, which may represent differentially glycosylated molecules (FIGS. 2 and 3, top panels). When the cells were co-transfected with corin expressing vector, both mouse and human AGRP protein levels were greatly reduced (FIGS. 2-4, top panels). In cell lysate samples, mouse and human AGRP proteins were expressed at similar levels in the presence or absence of recombinant corin (FIGS. 2-4, lower panels). The results indicated that the presence of corin did not inhibit AGRP protein expression inside the cell but promoted AGRP protein degradation in the conditioned medium.

Similar results were obtained for human ASIP protein. In the transfected HEK 293 cells, recombinant human ASIP protein was expressed, as indicated by a band of ˜21 kDa on Western blots (FIGS. 2 and 3, lower panels). The expression of this protein was not affected in the presence of recombinant corin protein. Recombinant human ASIP was secreted into the conditioned medium. Western analysis detected a band of ˜13 kDa (FIGS. 2 and 3, top panels). It appeared that an unknown enzyme in HEK 293 cells processed the protein into a mature peptide. In the presence of recombinant corin, the level of this processed human ASIP peptide was significantly reduced, suggesting that corin promotes human ASIP degradation.

As a positive control, recombinant corin converted human pro-ANP to ANP under similar experimental conditions (FIGS. 2-4, top panels). As a negative control, recombinant corin did not cleave human guanylin peptide nor alter its expression level in similarly transfected HEK 293 cells (FIG. 4).

Together, the data indicate that corin degrades, either directly or indirectly, AGRP and ASIP proteins. As AGRP and ASIP peptides play an important role in stimulating food intake, lack or low levels of corin may lead to higher AGRP and ASIP concentrations, thereby promoting more eating and causing obesity in animals and humans.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating obesity in an individual in need thereof comprising administering to the individual an effective amount of an agent that enhances corin expression, activity or a combination thereof in the individual.

2. The method of claim 1 wherein the agent is a polypeptide.

3. The method of claim 2 wherein the polypeptide is all or a biologically active portion of a mammalian corin protein.

4. The method of claim 3 wherein the mammalian corin protein is a human corin protein.

5. The method of claim 3 wherein the biologically active portion of the mammalian corin protein is a soluble corin polypeptide comprising all or a portion of the corin extracellular domain.

6. The method of claim 3 wherein the biologically active portion of the mammalian corin protein is a soluble corin polypeptide comprising the serine protease catalytic domain.

7. The method of claim 2 wherein the polypeptide is all or a biologically active portion of a modified mammalian corin protein.

8. The method of claim 7 wherein the modified corin protein comprises a protease recognition sequence other than the corin serine protease recognition sequence.

9. The method of claim 8 wherein the modified corin protein comprises a serine protease recognition sequence cleaved by a proteolytic enzyme selected from the group consisting of: enterokinase, thrombin, factor Xa, furin, PC1, PC2, PC5, PACE4 and a combination thereof.

10. The method of claim 7 wherein the biologically active portion of the modified corin protein comprises the corin extracellular domain and a protease recognition sequence other than the corin serine protease recognition sequence.

11. The method of claim 7 wherein the biologically active portion of the modified corin protein comprises the corin serine protease catalytic domain and a protease recognition sequence other than the corin serine protease recognition sequence.

12. The method of claim 1 wherein the agent is a nucleic acid.

13. The method of claim 14 wherein the nucleic acid encodes all or a biologically active portion of a mammalian corin protein.

14. The method of claim 13 wherein the nucleic acid is operably linked to the corin promoter.

15. The method of claim 13 wherein the mammalian corin protein is a human corin protein.

16. The method of claim 13 wherein the nucleic acid is administered as naked DNA or in an expression vector.

17. The method of claim 16 wherein the expression vector is a viral vector selected from the group consisting of: an adenoviral vector, a lentiviral vector, a poxviral vector.

18. The method of claim 13 wherein the biologically active portion of the mammalian corin protein is a soluble corin polypeptide comprising all or a portion of the corin extracellular domain.

19. The method of claim 13 wherein the biologically active portion of the mammalian corin protein is a soluble corin polypeptide comprising the serine protease catalytic domain.

20. The method of claim 13 wherein nucleic acid encodes all or a biologically active portion of a modified mammalian corin protein.

21. The method of claim 20 wherein the modified corin protein comprises a protease recognition sequence other than the corin serine protease recognition sequence.

22. The method of claim 21 wherein the modified corin protein comprises a serine protease recognition sequence cleaved by a proteolytic enzyme selected from the group consisting of: enterokinase, thrombin, factor Xa, furin, PC1, PC2, PC5, PACE4 and a combination thereof.

23. The method of claim 20 wherein the biologically active portion of the modified corin protein comprises the corin extracellular domain and a protease recognition sequence other than the corin serine protease recognition sequence.

24. The method of claim 20 wherein the biologically active portion of the modified corin protein comprises the corin serine protease catalytic domain and a protease recognition sequence other than the corin serine protease recognition sequence.

25. The method of claim 12 wherein the nucleic acid comprises all or a portion of the corin gene.

26. The method of claim 1 wherein the agent is a small organic molecule.

27. The method of claim 1 wherein the individual is a mammal.

28. The method of claim 27 wherein the mammal is a human.

29. The method of claim 1 wherein corin protein expression, activity or a combination thereof is increased in the individual after administration of the agent compared to corin protein expression, activity of a combination thereof in the individual prior to administration of the agent.

30. The method of claim 1 wherein the agent is a pharmaceutical agent.

31. A method of treating diabetes type II in an individual in need thereof comprising administering to the individual an effective amount of an agent that enhances corin expression, activity or a combination thereof in the individual.

32. The method of claim 31 wherein the agent is a polypeptide.

33. The method of claim 32 wherein the polypeptide is all or a biologically active portion of a mammalian corin protein.

34. The method of claim 33 wherein the mammalian corin protein is a human corin protein.

35. The method of claim 33 wherein the biologically active portion of the mammalian corin protein is a soluble corin polypeptide comprising all or a portion of the corin extracellular domain.

36. The method of claim 33 wherein the biologically active portion of the mammalian corin protein is a soluble corin polypeptide comprising the serine protease catalytic domain.

37. The method of claim 32 wherein the polypeptide is all or a biologically active portion of a modified mammalian corin protein.

38. The method of claim 37 wherein the modified corin protein comprises a protease recognition sequence other than the corin serine protease recognition sequence.

39. The method of claim 38 wherein the modified corin protein comprises a serine protease recognition sequence cleaved by a proteolytic enzyme selected from the group consisting of: enterokinase, thrombin, factor Xa, furin, PC1, PC2, PC5, PACE4 and a combination thereof.

40. The method of claim 37 wherein the biologically active portion of the modified corin protein comprises the corin extracellular domain and a protease recognition sequence other than the corin serine protease recognition sequence.

41. The method of claim 37 wherein the biologically active portion of the modified corin protein comprises the corin serine protease catalytic domain and a protease recognition sequence other than the corin serine protease recognition sequence.

42. The method of claim 31 wherein the agent is a nucleic acid.

43. The method of claim 34 wherein the nucleic acid encodes all or a biologically active portion of a mammalian corin protein.

44. The method of claim 33 wherein the nucleic acid is operably linked to the corin promoter.

45. The method of claim 33 wherein the mammalian corin protein is a human corin protein.

46. The method of claim 33 wherein the nucleic acid is administered as naked DNA or in an expression vector.

47. The method of claim 46 wherein the expression vector is a viral vector selected from the group consisting of: an adenoviral vector, a lentiviral vector, a poxviral vector.

48. The method of claim 43 wherein the biologically active portion of the mammalian corin protein is a soluble corin polypeptide comprising all or a portion of the corin extracellular domain.

49. The method of claim 43 wherein the biologically active portion of the mammalian corin protein is a soluble corin polypeptide comprising the serine protease catalytic domain.

50. The method of claim 43 wherein nucleic acid encodes all or a biologically active portion of a modified mammalian corin protein.

51. The method of claim 50 wherein the modified corin protein comprises a protease recognition sequence other than the corin serine protease recognition sequence.

52. The method of claim 51 wherein the modified corin protein comprises a serine protease recognition sequence cleaved by a proteolytic enzyme selected from the group consisting of: enterokinase, thrombin, factor Xa, furin, PC1, PC2, PC5, PACE4 and a combination thereof.

53. The method of claim 50 wherein the biologically active portion of the modified corin protein comprises the corin extracellular domain and a protease recognition sequence other than the corin serine protease recognition sequence.

54. The method of claim 50 wherein the biologically active portion of the modified corin protein comprises the corin serine protease catalytic domain and a protease recognition sequence other than the corin serine protease recognition sequence.

55. The method of claim 32 wherein the nucleic acid comprises all or a portion of the corin gene.

56. The method of claim 31 wherein the agent is a small organic molecule.

57. The method of claim 31 wherein the individual is a mammal.

58. The method of claim 57 wherein the mammal is a human.

59. The method of claim 31 wherein corin protein expression, activity or a combination thereof is increased in the individual after administration of the agent compared to corin protein expression, activity of a combination thereof in the individual prior to administration of the agent.

60. The method of claim 31 wherein the agent is a pharmaceutical agent.
